(19) United States
(12) Patent Application Publication
(10) Pub. No.: US 2006/0178542 A1
(43) Pub. Date: Aug. 10, 2006

(54) Aromatic ring polymer and low-dielectric material

(76) Inventors: Mitsuru Ueda, Tokyo (JP); Hirotoshi Ishii, Sodegaura-shi (JP)

Correspondence Address: STEPTOE & JOHNSON LLP, 1330 CONNECTICUT AVENUE, N.W., WASHINGTON, DC, 20036, US

(21) Appl. No.: 10/548,963

(22) PCT Filed: Jul. 31, 2003
(86) PCT No.: PCT/JP03/09723

§ 371 Date: Sep. 15, 2005

(30) Foreign Application Priority Data
Mar. 17, 2003 (JP) 2003-116342
Mar. 19, 2003 (JP) 2003-119086
May 1, 2003 (JP) 2003-161377

(51) Int. Cl. C07C 15/02; C07C 403/00; B32B 9/04; B32B 19/00

(52) U.S. Cl. 585/400; 428/690; 428/411.1; 428/901

(57) ABSTRACT

An aromatic ring polymer wherein adjacent aromatic ring skeletons cannot have a conformation positioned on a single plane due to mutual steric hindrance of the adjacent aromatic ring skeletons and is represented by the following formula (1): [symbol]
X-A-Y-B [symbol] ₙ (1) wherein X and Y may be the same or different, and each represent a monocyclic or heterocyclic bivalent aromatic group which may be substituted with R; A and B may be the same or different, and are each a single bond or a substituent which can contain an aromatic group; and n is an integer of 5 to 1000000. This polymer has a low dielectric constant, and is excellent in heat resistance and strength.

### TECHNICAL FIELD

The present invention relates to a new aromatic ring polymer which is useful as low dielectric material, heat resistant material or high strength material in electrical and electronic fields. The invention relates in particular to an interlayer dielectric film material for semiconductor.

### BACKGROUND ART

A low dielectric material has widely been used as a material in electrical and electronic parts in order to overcome problems such as electrification and a rise in resistance. In many cases, a low dielectric material is used in portions where heat is generated or portions where stress is concentrated, or is used as a thin film. Accordingly, a low dielectric material has been required to have not only a low dielectric constant but also an improved heat resistance and strength. A low dielectric material is used, in particular, as an interlayer dielectric film material for semiconductor. Thus, a material having a low dielectric constant, a high heat resistance, a high strength and economy has been actively developed.

At present, siloxane compounds have mainly been used as the material of the interlayer dielectric film for semiconductor, which is a main intended application of low dielectric material. Siloxane compounds are made mainly of silicon and oxygen. As the dipole moment of the molecule of a siloxane compound is larger, the dielectric constant thereof is higher; therefore, siloxane compounds having many free electron pairs are unfavorable for a low dielectric material. However, siloxane compounds have been used from the viewpoint of the balance between strength and adhesive property to silicon wafers since dielectric constant values which have been hitherto required are from about 4 to 3 (=k).

In recent years, the width of semiconductor circuits has been desired to be made finer on the basis of a request that the performance of semiconductors is made higher. Thus, it has become necessary to make the dielectric constant thereof lower. In this case, problems about the strength of the whole of a semiconductor chip and about dielectric breakdown based on physical stress and others become serious. It is therefore necessary to keep the strength thereof required as a thin film. From the viewpoint of a drop in the dielectric constant of siloxane compounds, technique has been advancing from inorganic siloxane compounds through organic siloxane compounds to the compounds where pores controlled into a nanometer level are introduced.

However, if the amount of the introduced pores is increased to further reduce the dielectric constant, a drop in the strength becomes a problem. Thus, new materials such as organic polymers have been suggested. However, there has been found no material having, in particular, a high heat resistance such that thermal load imposed at the time of semiconductor-production can be resisted, as well as insulation, a low dielectric constant and a high strength. Organic/inorganic polymers, such as borazine-silicon based polymer, have also been suggested. The polymer has a low dielectric constant, a high strength and a high heat resistance; however, problems remain about dielectric breakdown and a low stability generated by remaining platinum atoms since there is no step of removing any platinum catalyst necessary for the polymerization (see, for example, Japanese Patent Application Laid-Open No.2002-359240).

As described above, in order to reduce the dielectric constant of interlayer dielectric film materials known in the prior art, the amount of introduced pores of a nanometer level size is increased. However, the increase in the amount of the introduced pores causes a fall in the strength. That is, there is a limit to the fall in the dielectric constant without lowering the strength.

The above-mentioned compounds are used as a surface protecting film also. However, the compounds are used as thermally crosslinking materials; therefore, the compounds need to be subjected to thermal treatment at high temperature in order to exhibit a desired performance. A material for which thermal treatment is unnecessary has been desired from the viewpoint of the prevention of damage onto devices and others, based on the thermal treatment, and economy.

An object of the present invention is to overcome various problems generated by an increase in the amount of pores introduced into interlayer dielectric film material using a low dielectric material known in the prior art, and provide a low dielectric material excellent for interlayer dielectric film material for which the introduction of pores is unnecessary.

There is a limit to a fall in the dielectric constant, without lowering the strength, according to current methods of introducing pores of a nanometer level size. It is therefore necessary to introduce pores of an angstrom level size. It is nothing else but an increase in pores of an atomic size, that is, in intermolecular free volume. A specific structure of such a material has been devised, so that the present invention has been completed.

Another object of the invention is to provide a heat resistant material which can exhibit a high heat resistance without requiring any thermal crosslinking.

Still another object of the invention is to provide a high strength material which can exhibit a high strength without requiring any thermal crosslinking.

### DISCLOSURE OF THE INVENTION

According to the present invention, the following aromatic ring polymer, and so on can be provided:
[1] An aromatic ring polymer, comprising a main chain in which aromatic rings range, the aromatic ring polymer having a dipole moment of 1 debye or less and/or a density of 1.50 g/cm³ or less by cancellation of the dipole moments of the aromatic rings in the most stable structure thereof.
[2] An aromatic ring polymer, wherein adjacent aromatic ring skeletons cannot have a conformation positioned on a single plane due to mutual steric hindrance of the adjacent aromatic ring skeletons and is represented by the following formula (1): [symbol]
   X-A-Y-B [symbol] ₙ (1)
   wherein X and Y may be the same or different, and each represent a monocyclic or heterocyclic aromatic group which may be substituted with R;

A and B may be the same or different, and each represent a single bond, a bifunctional substituent selected from —(CR₂)ₘ—, —(SiR₂)ₘ—, —(OSiR₂O)ₘ—, —(SiRO_{1.5})ₘ—, —(GeR₂)ₘ—, —(SnR₂)ₘ—, —BR—, —AlR—, —NR—, —PR—, —AsR—, —SbR—, —O—, —S—, —Se—, —Te—, —CO—, —COO—, —OO—, —NHCO—, —(N═C)—, an acetylidene group, an ethylidene group, a borazilene group, a substituted or unsubstituted aromatic group having 6 to 50 carbon atoms and a substituted or unsubstituted heteroatom-containing aromatic group having 4 to 50 carbon atoms, or a substituent formed by combining one or more out of these substituents;

R may be the same or different, and each represent an alkyl group having 1 to 20 carbon atoms, an alkenyl group having 1 to 20 carbon atoms, an alkynyl group having 1 to 20 carbon atoms, a substituted or unsubstituted aromatic group having 6 to 20 carbon atoms, an ether group, a thioether group, an ester group, an epoxy-containing group, a silyl-containing group, a siloxy-containing group, a fluorine-containing group, a borazyl group, or a substituent formed by combining two or more out of these substituents;

m is an integer of 1 to 50; and

n is an integer of 5 to 1000000.
[3] An aromatic ring polymer, wherein adjacent aromatic ring skeletons cannot have a conformation positioned on a single plane due to mutual steric hindrance of the adjacent aromatic ring skeletons and is represented by the following formula (2): [symbol]
   X-A′-Y [symbol] ₙ (2)
   wherein X and Y may be the same or different, and each represent a monocyclic or heterocyclic aromatic group which may be substituted with R;

A′ may be the same or different, and each represent a monocyclic or heterocyclic aromatic group which may be substituted with R and is bonded to X and Y through any one of oxygen, nitrogen, sulfur, silicon and boron or through a substituent containing one or more out of these atoms;

R may be the same or different, and each represent an alkyl group having 1 to 20 carbon atoms, an alkenyl group having 1 to 20 carbon atoms, an alkynyl group having 1 to 20 carbon atoms, a substituted or unsubstituted aromatic group having 6 to 20 carbon atoms, an ether group, a thioether group, an ester group, an epoxy-containing group, a sily 1-containing group, a siloxy-containing group, a fluorine-containing group, or a substituent formed by combining two or more out of these substituents; and

n is an integer of 5 to 1000000.
[4] An aromatic ring polymer represented by the following formula (3): 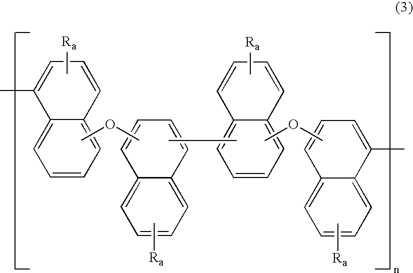
   wherein each R and n are the same as in the formula (1), and a may be the same or different, and are each an integer of 0 to 6.
[5] An aromatic ring polymer represented by the following formula (4): 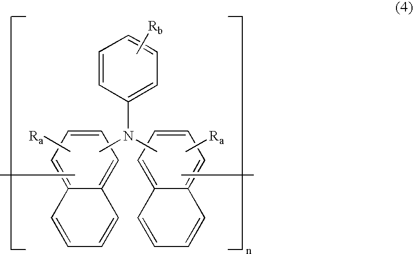
   wherein each R and n are the same as in the formula (1), a may be the same or different, and are each an integer of 0 to 6, and b may be the same or different, and are each integer of 0 to 5.
[6] The aromatic ring polymer according to any one of items [2] to [5], which has a dipole moment of 1 debye or less, and/or a density of 1.20 g/cm³ or less.
[7] A low dielectric material, composed of the aromatic ring polymer according to any one of items [1] to [6].
[8] An interlayer dielectric film for semiconductor, composed of the low dielectric material according to item [7].
[9] A heat resistant material, composed of the aromatic ring polymer according to any one of items [1] to [6].
[10] The heat resistant material according to item [9], the glass transition temperature of the heat resistant material being 250° C. or higher, and a lower temperature out of the melting temperature thereof or the thermal decomposition starting temperature thereof being 300° C. or higher.
[11] A high strength material, composed of the aromatic ring polymer according to any one of items [1] to [6].
[12] The high strength material according to item [11], which has a hardness of 0.3 GPa or more, and/or a modulus of 3 GPa or more.
[13] A thin film, composed of the aromatic ring polymer according to any one of items [1] to [6].
[14] A semiconductor device, composed of the thin film according to item [13].
[15] An image display device, composed of the thin film according to item [13].
[16] An electronic circuit device, composed of the thin film according to item [13].
[17] A surface protecting film, composed of the thin film according to item [13].
[18] A paint wherein the aromatic ring polymer according to any one of items [1] to [6] is dissolved in an organic solvent.

### BRIEF DESCRIPTION OF THE DRAWINGS

FIG. 1 is a view illustrating an embodiment of the semiconductor device of the present invention.

FIG. 2 is a chart of ¹H-NMR of 2,2′-dinaphthyloxy-1,1′-binaphthyl yielded in Production Example 1.

FIG. 3 is a chart of ¹³C-NMR of 2,2′-dinaphthyloxy-1,1′-binaphthyl yielded in Production Example 1.

FIG. 4 is a chart of ¹H-NMR of di-(1-naphthyl)-4-toluylamine in Production Example 2.

FIG. 5 is a chart of ¹³C-NMR of di-(1-naphthyl)-4-toluylamine in Production Example 2.

FIG. 6 is a chart of ¹H-NMR of poly(2,2′-dinaphthyloxy-1,1′-binaphthyl) in Example FIG. 7 is a chart of ¹³C-NMR of poly(2,2′-dinaphthyloxy-1,1′-binaphthyl)in Example 1.

FIG. 8 is a chart of ¹H-NMR of poly(di-(1-naphthyl)-4-toluylamine) in Example 2.

FIG. 9 is a chart of ¹³C-NMR of poly(di-(1-naphthyl)-4-toluylamine) in Example 2.

### BEST MODE FOR CARRYING OUT THE INVENTION

The present invention is an aromatic ring polymer which has a main chain wherein aromatic rings range and which has a dipole moment of 1 debye or less and/or a density of 1.50 g/cm³ or less by cancellation of the dipole moments of the aromatic rings in the most stable structure thereof.

The aromatic rings may be the same or different, and are each a substituted or unsubstituted monocyclic or heterocyclic aromatic group, such as a naphthalene ring or a benzene ring.

The most stable structure means the structure obtained by performing structure optimization in the AM1 manner of a semi-experimental orbital method program package, MOPAC 97.

The dipole moment can be obtained from the most stable structure by theoretical calculation.

The directions of the dipole moments of a large number of the aromatic rings present in the main chain of this polymer are not consistent with each other; therefore, the dipole moments are cancelled out so that the dipole moment of the whole of the polymer becomes 1 debye or less, preferably 0.7 debye or less.

The value of the dipole moment can be adjusted with the kind of the aromatic rings and that of substituent (s) in the aromatic rings, the substitution position thereof, and the number of the substituent(s).

The value can be adjusted, for example, by lowering the concentration of free electron pairs contained in the molecule. However, if the concentration is too low, the workability generally deteriorates. Thus, the value is preferably set into 0.01 debye or more.

The density can be obtained by making the polymer into a thin film having no pores of 2 nm or more size, and then measuring the thin film by the oblique incident X-ray reflectance method.

The polymer of the present invention has a geometrically large intermolecular free volume on the basis of the steric repulsion and the twisted structure of the aromatic ring structure.

Like the dipole moment, the density can be adjusted with the kind of the aromatic rings and that of substituent(s) in the aromatic rings, the substitution position thereof, and the number of the substituent (s) The density can be adjusted preferably into 1.50 g/cm³ or less, more preferably 1.20 g/cm³ or less.

In such a polymer, the main chain thereof is twisted by steric repulsion of the aromatic ring structure, thereby resulting in low dielectricity. In other words, the dipole moments of a large number of the aromatic rings are randomized so as to be cancelled out, thereby generating a large intermolecular free volume.

The present invention is also an aromatic ring polymer where adjacent aromatic ring skeletons cannot have a conformation positioned on a single plane due to mutual steric hindrance of the adjacent aromatic ring skeletons and is represented by the following formula (1): [symbol]
X-A-Y-B [symbol] ₙ (1)
wherein X and Y may be the same or different, and each represent a monocyclic or heterocyclic aromatic group which may be substituted with R;

A and B may be the same or different, and each represent a single bond, a bifunctional substituent selected from —(CR₂)ₘ—, —(SiR₂)ₘ—, —(OSiR₂O)ₘ—, —(SiRO_{1.5})ₘ—, —(GeR₂)ₘ—, —(SnR₂)ₘ—, —BR—, —AlR—, —NR—, —PR—, —AsR—, —SbR—, —O—, —S—, —Se—, —Te—, —CO—, —COO—, —OO—, —NHCO—, —(N═C)—, an acetylidene group, an ethylidene group, a borazilene group, a substituted or unsubstituted aromatic group having 6 to 50 carbon atoms and a substituted or unsubstituted heteroatom-containing aromatic group having 4 to 50 carbon atoms, or a substituent formed by combining one or more out of these substituents;

R may be the same or different, and each represent an alkyl group having 1 to 20 carbon atoms, an alkenyl group having 1 to 20 carbon atoms, an alkynyl group having 1 to 20 carbon atoms, a substituted or unsubstituted aromatic group having 6 to 20 carbon atoms, an ether group, a thioether group, an ester group, an epoxy-containing group, a silyl-containing group, a siloxy-containing group, a fluorine-containing group, a borazyl group, or a substituent formed by combining two or more out of these substituents;

m is an integer of 1 to 50; and

n is an integer of 5 to 1000000.

In this aromatic ring polymer, adjacent aromatic ring skeletons therein are not positioned on a single plane in the most stable structure obtained in the AM1 manner of the above-mentioned semi-experimental orbital method program, MOPAC 97. For this reason, the dipole moments of the aromatic ring skeletons are cancelled out to generate a large intermolecular free volume. It appears that this aromatic ring polymer therefore has a low dielectric constant.

The aromatic ring skeletons are aromatic ring skeletons contained in X, A, Y and B.

Preferred examples of the aromatic ring polymer include the following:

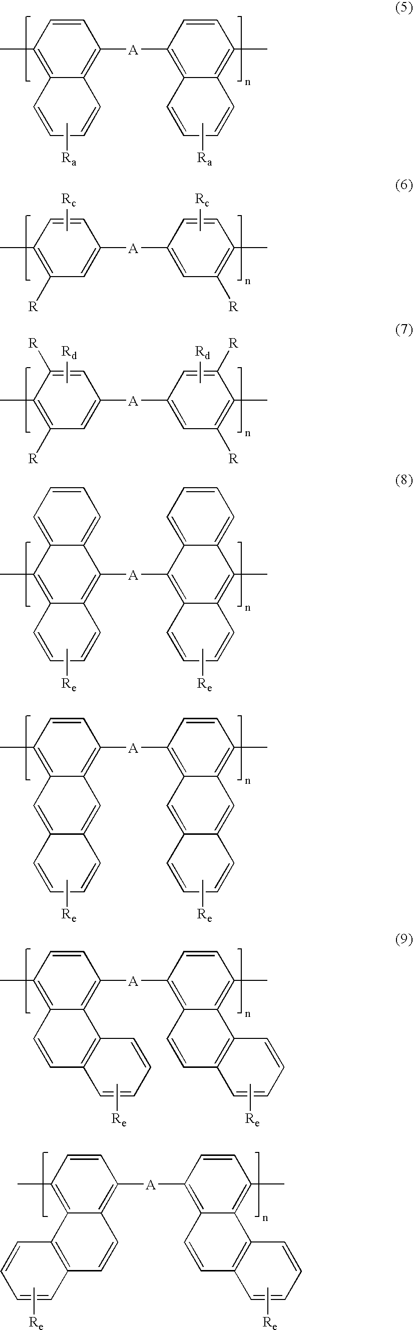

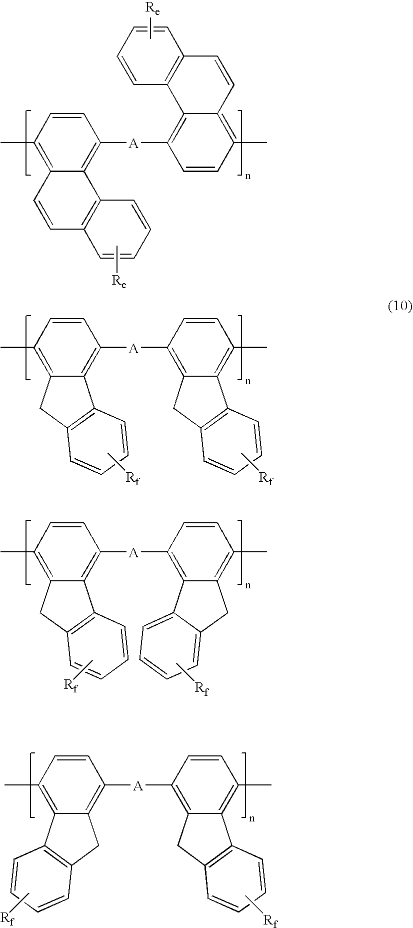

In the formulae (5) to (10), A, R and n are the same as in the formula (1).

In the formula (5), a may be the same or different, and are each an integer of 0 to 6.

In the formula (6), c may be the same or different, and are each an integer of 0 to 3.

In the formula (7), d may be the same or different, and are each an integer of 0 to 2.

In the formulae (8) and (9), e may be the same or different, and are each an integer of 0 to 8.

In the formula (10), f may be the same or different, and are each an integer of 0 to 8.

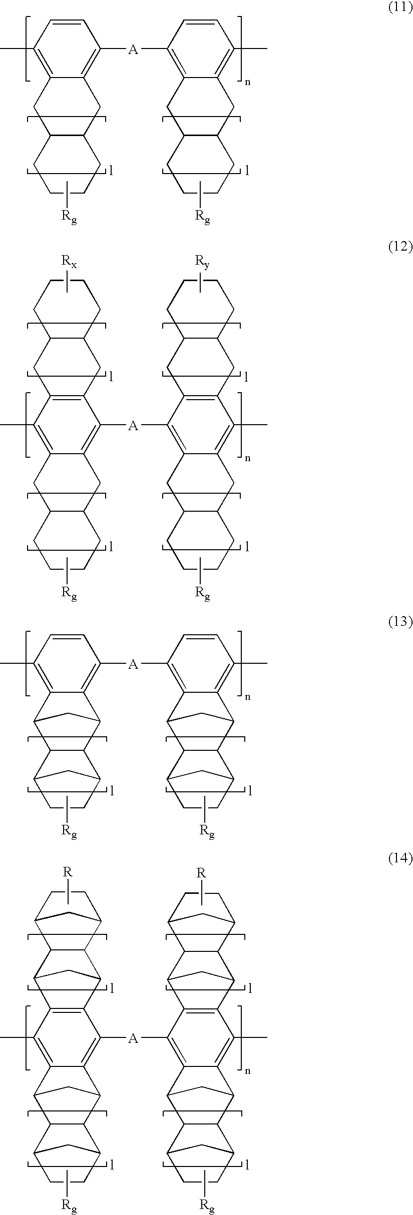

In the formulae (11) to (14), A, R and n are the same as in the formula (1), l may be the same or different, and are each an integer of 0 to 5, and g may be the same or different, and are each an integer of 0 to 38 which satisfies the relationship of g=1×6+8.

In the formulae (11) to (14), when 1 are each an integer of 1 to 5, arbitrary isomers where the substitution positions of the cyclohexane ring or the norbornene ring structures bonded to each other in a condensed ring form are various are not excluded.

Preferred aromatic groups of X and Y are a naphthalene or benzene ring.

A is preferably a monocyclic or heterocyclic aromatic group which may be substituted with R and is bonded to X and Y through any one of oxygen, nitrogen, sulfur, silicon and boron or through a substituent containing one or more out of these atoms. A is more preferably a binaphthyl ring, benzene ring or biphenyl ring which may be substituted with R and is bonded to X and Y through any one of oxygen, nitrogen and sulfur or through any one of oxygen, nitrogen, sulfur, silicon and boron.

B is preferably a single bond.

n is preferably an integer of 5 to 100000, in particular preferably an integer of 5 to 5000.

In the formula (1), examples of each R include alkyl groups having 1 to20 carbon atoms, such as methyl, ethyl, n-propyl, iso-propyl, cyclopropyl, n-butyl, iso-butyl, tert-butyl, 2-ethylhexyl, n-decyl, n-dodecyl, cyclohexyl, norbornyl, adamantyl, and biadamantyl groups; alkenyl groups having 1 to 20 carbon atoms, such as vinyl, isopropenyl, and allyl groups; alkynyl groups having 1 to 20 carbon atoms, such as an ethynyl group; aromatic groups having 6 to 20 carbon atoms, such as phenyl, naphthyl, anthracenyl, and phenanthrenyl groups; aromatic groups which have 6 to 20 carbon atoms and are substituted with an alkyl group having 1 to 20 carbon atoms, such as toluyl and cumyl groups; alkoxy groups having 1 to 20 carbon atoms, such as methoxy, ethoxy, adamantyloxy and biadamantyloxy groups, alkenyloxy groups having 1 to 20 carbon atoms, such as vinyloxy and allyloxygroups, aphenoxy group; alkylthio groups having 1 to 20 carbon atoms, such as methylthio and adamantylthio groups, alkenylthio groups having 1 to20carbon atoms, suchasavinylthio group, a phenylthio group; ester groups such as acetoxy, acryloxy and methacryloxy groups; an epoxy group, alkylepoxy groups having 1 to 20 carbon atoms, such as an epoxymethyl group; a silyl group, trialkylsilyl groups such as trimethylsilyl and tert-butyldimethylsilyl groups, a triphenylsilyl group; a siloxy group, trialkylsiloxy groups such as trimethylsiloxy and tert-butyldimethylsiloxy groups, a triphenylsiloxy group; fluorine, fluorinated alkyl groups having 1 to 20 carbon atoms, such as a trifluoromethyl group, fluorinated alkenyl groups having 1 to 20 carbon atoms, such as a hexafluoroisopropenyl group, a pentafluorophenyl group, fluorinated alkoxy groups having 1 to 20 carbon atoms, such as a trifluoromethoxy group, fluorinated alkenyloxy groups having 1 to 20 carbon atoms, such as a hexafluoroisopropenoxy group, a pentafluorophenoxy group; and substituents formed by combining two or more out of the above-mentioned substituents, such as p-trifluoromethylphenyl, p-trifluoromethylphenoxy, vinyladamantyl, vinyladamantyloxy and vinylbiadamantyloxy groups.

Preferred examples of each R include methyl, ethyl, cyclopropyl, n-butyl, tert-butyl, n-dodecyl, cyclohexyl, norbornyl, adamantyl, biadamantyl, vinyl, isopropenyl, allyl, ethynyl, phenyl, naphthyl, anthracenyl, phenanthrenyl, toluyl, cumyl, methoxy, ethoxy, phenoxy, adamantyloxy, biadamantyloxy, vinyloxy, allyloxy, adamantylthio, vinylthio, acryloxy, methacryloxy, epoxy, epoxymethyl, trimethylsilyl, triphenylsilyl, trimethylsiloxy, triphenylsiloxy, trifluoromethyl and trifluoromethoxy groups, and fluorine.

Particularly preferred examples of each R include methyl, n-butyl, tert-butyl, adamantyl, biadamantyl, vinyl, isopropenyl, allyl, ethynyl, phenyl, naphthyl, anthracenyl, phenanthrenyl, methoxy, phenoxy, adamantyloxy, biadamantyloxy, vinyloxy, allyloxy, adamantylthio, vinylthib, acryloxy, methacryloxy, trimethylsilyl, triphenylsilyl, trimethylsiloxy, triphenylsiloxy, trifluoromethyl and trifluoromethoxy groups, and fluorine.

The following will specifically describe the aromatic ring polymer of the formula (1). Preferred examples of each R and n in the following formulae are the same as described above.

An example of the aromatic ring polymer of the formula (1) is an aromatic ring polymer represented by the following formula (3): 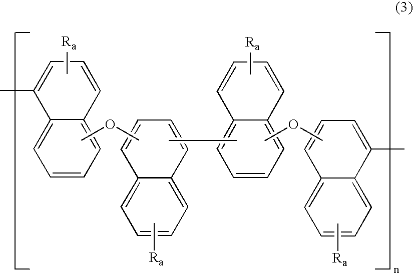
wherein each R and n are the same as in the formula (1) and a may be the same or different, and are each an integer of 0 to 6.

As shown in the formula, bonding positions of each of the naphthalene rings and the substitution position of each R are not particularly limited. Each a is the number of R in each of the naphthalene rings, and is an integer of 0 to 6 in each of the naphthalene rings.

Each a is preferably an integer of 0 to 4, more preferably an integer of 0 to 1.

A preferred example of the polymer of the formula (3) is an aromatic ring polymer represented by the following formula (15): 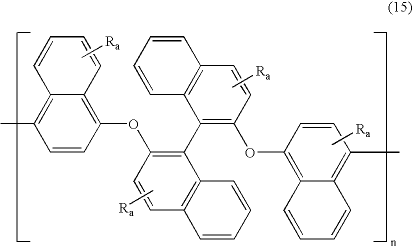
wherein each R, each a, and n are the same as in the, formula (.3).

Another example of the aromatic ring polymer of the formula (1) is an aromatic ring polymer represented by the following formula (4): 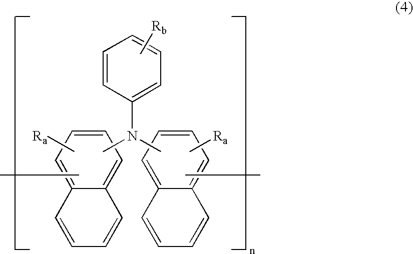
wherein each R, each a, and n are the same as in the formula (1), and b may be the same or different, and are each an integer of 0 to 5.

As shown in this formula, the bonding position between the two naphthalene rings and the benzene ring, and the substitution position of each R are not particularly limited. Each a is the number of R in each of the naphthalene rings, and is an integer of 0 to 6 in each of the naphthalene rings b is the number of R in the benzene ring, and is an integer of 0 to 5.

A preferred example of the polymer of the formula (4) is a dinaphthylamine polymer represented by the following formula (16): 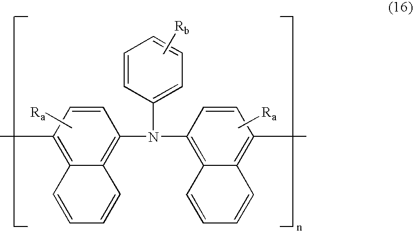
wherein each R, each a, b, and n are the same as in the formula (4).

The following will describe a process for producing the above-mentioned aromatic ring polymer.

The aromatic ring polymer of the formula (1) can be produced by polymerizing a monomer represented by the following formula (17):
X-A-Y-B (17)
wherein X, A, Y and B are the same as in the formula (1). Preferably, the polymerization is oxidation polymerization.

The aromatic ring polymer of the formula (3) can be synthesized by polymerizing a monomer represented by the following formula (18): 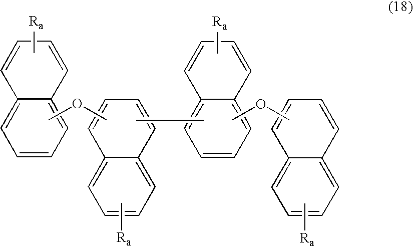
wherein each R and each a are the same as in the formula (3). Preferably, the polymerization is oxidation polymerization.

The aromatic ring polymer of the formula (15) is synthesized by polymerizing a monomer represented by the following formula (19): 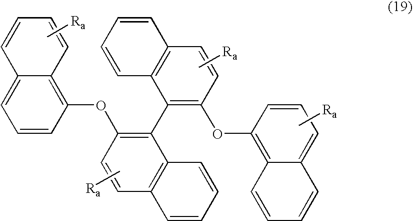
wherein each R and each a are the same as in the formula (15).

The method for the oxidation polymerization of the above-mentioned monomer is not particularly limited, and is generally known. Examples thereof include a method of carrying out the polymerization in a suspension of ferric chloride in the atmosphere of nitrogen gas, and a method of using a vanadyl oxide as a catalyst and trifluoroacetic anhydride as a dehydrating agent in trifluoroacetic acid and introducing oxygen.

The monomer of the formula (18) can be synthesized by a reaction known in the prior art, such as dehydrating reaction, Williamson reaction, Ullmann reaction or Mitsunobu reaction, using as starting materials one or more selected from binaphthyls represented by the following formula (20):. 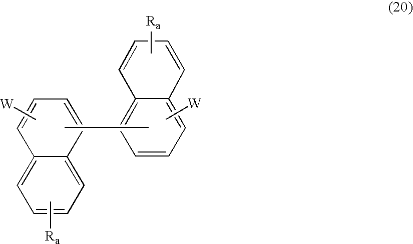
wherein each R and each a are the same as in the formula (18), and W are each a substituent active in ether synthesizing reaction, such as a hydroxyl group, bromine, chlorine or iodine, and one or more selected from naphthalenes represented by the following formula (21): 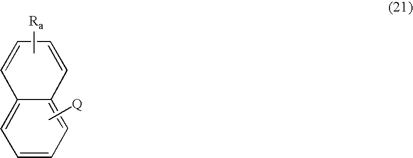
wherein R and a are the same as in the formula (18), and Q is a substituent active in ether synthesizing reaction, such as a hydroxyl group, bromine, chlorine or iodine.

However, at least one of W in the formula (20) and Q in the formula (21) is hydroxyl.

The monomer of the formula (19) can be synthesized in the same way, using as starting materials one or more selected from 1,1′-binaphthyls represented by the following formula (22): 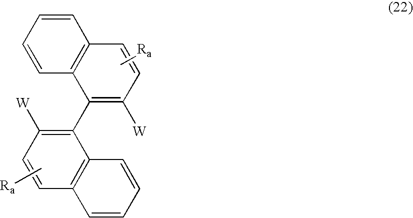
wherein each R and each a are the same as in the formula (19), and W are each a substituent active in ether synthesizing reaction, such as a hydroxyl group, bromine, chlorine or iodine, and one or more selected from naphthalenes represented by the following formula (23): 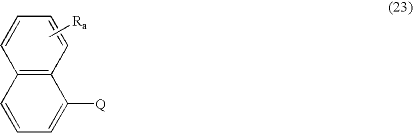
wherein R and a are the same as in the formula (19), and Q is a substituent active in ether synthesizing reaction, such as a hydroxyl group, bromine, chlorine or iodine.

However, at least one of W's in the formula (22) and Q in the formula (23) is hydroxyl.

Specific examples of the 1,1′-binaphthyls represented by the formula (22), wherein W are bonded to its 2 and 2′ positions, include 2,2′-dihydroxy-1,1′-binaphthyl, 2,2′-dichloro-1,1′-binaphthyl, 2,2′-dibromo-1,1′-binaphthyl, 2,2′-diiode-1,1′-binaphthyl, and 1,1′-binaphthyls wherein these have R, the number of which is a.

Specific examples of the naphthalenes represented by the formula (23), wherein Q is bonded to its 1 position, include 1-naphthol, 1-chloronaphthalene, 1-bromonaphthalene, 1-iodonaphthalene, and naphthalenes where these have R, the number of which is a.

The monomers of the formulae (20) to (23) can be obtained as commercially available products, or can be produced by known methods.

The dinaphthylamine polymer of the formula (4) can be synthesized by polymerizing a monomer represented by the following formula (24): 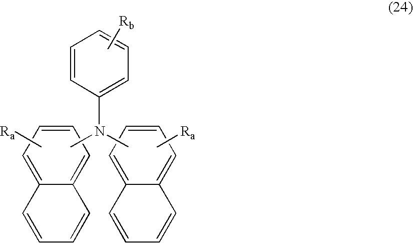
wherein each R, each a, and b are the same as in the formula (4). The polymerization is preferably oxidation polymerization.

The dinaphthylamine polymer of the formula (16) is synthesized by polymerizing a monomer represented by the following formula (25): 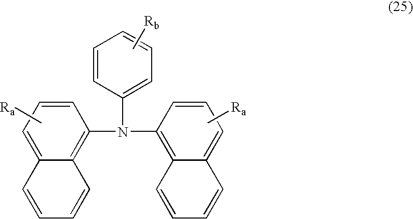
wherein each R, each a, and b are the same as in the formula (16).

The monomer of the formula (24) can be synthesized by a reaction known in the prior art, such as an amine compound arylating reaction, in the presence of a catalyst selected from palladium compounds, nickel compounds, copper compounds and ruthenium compounds and/or a base, using as starting materials one or more selected from phenylamines represented by the following formula (26): 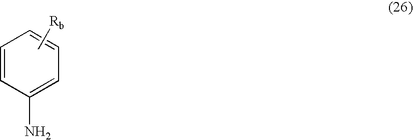
wherein R and b are the same as in the formula (24), and
one or more selected from naphthalenes represented by the following formula (27): 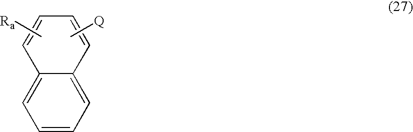
wherein R and a are the same as in the formula (24), and Q is a substituent active in amine arylating reaction, such as fluorine, chlorine, bromine, iodine, a hydroxyl group, or an organic boron group.

The monomer of the formula (25) can be synthesized in the same way, using as starting materials one or more selected from phenylamines represented by the formula (26), and one or more selected from naphthalenes represented by the following formula (28): 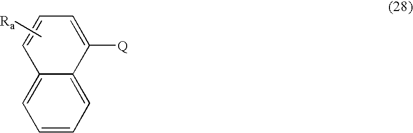
wherein R and a are the same as in the formula (25), and Q is a substituent active in amine arylating reaction, such as fluorine, chlorine, bromine iodine, a hydroxyl group, or an organic boron group.

Examples of the phenylamines represented by the formula (26) include 4-methylphenylamine, 3,5-dimethylphenylamine, 4-adamantylphenylamine, 4-adamantyloxyphenylamine, 4-biadamantylphenylamine, 4-biadamantyloxyphenylamine, and phenylamine.

Examples of the naphthalenes represented by the formula (28), wherein Q is bonded to its 1 position, include 1-naphthol, 1-chloronaphthalene, 1-bromonaphthalene, 1-iodonaphthalene, and naphthalenes where these have R, the number of which is a.

The monomers of the formulae (26) to (28) can be obtained as commercially available products, or can be produced by known methods.

The dipole moment and the density of the aromatic ring polymer of the present invention are varied in accordance with the kind of the aromatic rings and that of the substituent(s) R, the substitution position thereof, and the number of the substituent(s). The dipole moment and the density are preferably 1 debye or less and 1.50 g/cm³ or less, respectively.

The aromatic ring polymer can be used as low dielectric material of various electrical or electronic parts, in particular, as interlayer dielectric film material for semiconductor that is used for semiconductor devices since the polymer has a low dielectric constant.

In particular, in the aromatic ring polymer of the formula (3), the binaphthyl group sandwiched between two naphthalene rings across ether bonds is bulky and is not easily free-rotated because of steric hindrance thereof. Similarly, in the aromatic ring polymer of the formula (4), the substituted benzene group sandwiched between two naphthalene rings is bulky and is not easily free-rotated because of steric hindrance thereof. Furthermore, the naphthalene rings ranging in the main chain are arranged in different directions; therefore, the dipole moments measured by the molecular orbital method are cancelled out so that a specific dipole moment is not easily formed. It appears that this fact make the dielectric constant thereof low.

The dielectric constant of the aromatic ring polymer of the present invention, which is in accordance with the kind of the aromatic rings, that of the substituent(s) R, the substitution position thereof and the number of the substituent(s), is preferably 3.0 or less, more preferably 2.7 or less, even more preferably 2.5 or less as the value of k. Even if the polymer has no substituent, the dipole moments are randomized and cancelled out by the twist of the main chain based on steric repulsion of the aromatic ring structures, so as to lower the dipole moment of the whole of the molecule and further the polymer has a geometrically large intermolecular free volume on the basis of the steric repulsion and the twisted structure of the aromatic ring structures. Accordingly, the polymer exhibits a lower dielectric constant than common polyarylenes such as polyphenylene. The steric repulsion and the dipole moments of the aromatic ring structures can be appropriately adjusted with the kind of the substituent(s) R therein, the substitution position thereof, and the number of the substituent(s).

The interlayer dielectric film material of ULSI multi-layered wiring structure in semiconductor-production is required to have the properties such as dielectricity, heat resistance, strength, adhesive property to a substrate, and stability. These properties are varied in accordance with the number of layers in the used multi-layered wiring or design nodes; thus, specific values thereof cannot be specified. In general, it is desired that the dielectricity is low and the heat resistance, the strength, the adhesive property to a substrate, the stability, and so on are high. The aromatic ring polymer of the present invention has these properties.

As described above, the aromatic ring polymer of the invention can be favorably used as an interlayer dielectric film for semiconductor devices since the polymer has a low dielectric constant. However since the polymer also has excellent in other properties, such as a high heat resistance, the polymer can be used as a different member in semiconductor devices, image display devices, electronic circuit devices, or the like.

The aromatic ring polymer used in the invention can be used as a heat resistant material for various electrical or electronic parts because the polymer has a high heat resistance.

When the heat resistant material of the invention is used, heat resistance is given to various articles, typical examples of which are semiconductors such as a ULSI, without any thermal treatment. As a result, the performance or reliability thereof can greatly be improved. It is probable that the aromatic ring polymer used in the invention has a high heat resistance since the polymer has the following molecular structure:

(1) Aromatic ring structures in which it is difficult to generate radicals by heat or wherein even if radicals are generated, the radicals are stably present so that it is difficult to cause isomerization reaction or the like.

(2) A steric structure having a main chain in which radical coupling inside the molecule or coupling between the molecules does not advance easily.

(3) A structure in which each molecular structure is relatively rigid and further aromatic ring π electron electrostatic interaction of the aromatic ring structures and intermolecular interaction based on zigzag structure are intense so that the packing state of the molecule is not easily changed by heat.

The method for evaluating the heat resistance can be attained by an ordinary thermoproperty evaluation, such as a differential scanning calorimeter (DSC) or a thermogravimetry differential thermal analyzer (Tg/DTA). The form of a sample for the evaluation may be a thin film, powder which is a precursor thereof, or a block, and can be appropriately selected depending on the requirements of a device used for the evaluation. The heat resisting temperature is prescribed with two kinds of temperatures: the glass transition temperature, and a lower temperature out of the melting temperature and the thermal decomposition starting temperature, each of which is obtained by the above-mentioned method.

The glass transition temperature is varied in accordance with X, Y, A and B, which are main chain structures of the formula (1), the kind of the substituent(s) R, the substitution position thereof, the number of the substituent (s), the molecular weight, the molecular weight distribution, and so on, and is preferably 250° C. or higher, more preferably 300° C. or higher. The lower temperature out of the meting temperature and the thermal decomposition starting temperature is varied in accordance with the kind of the substituent(s) R, the substitution position thereof, and the number of the substituent(s), and is preferably 300° C. or higher, more preferably 400° C. or higher. The polymer used in the invention is seldom decomposed by radicals generated by heat and has a high heat resistance since the polymer is a kind of polyarylene.

The aromatic ring polymer used in the invention can be used as high strength material for various electrical or electronic parts.

When the high strength material of the invention is used, high strength is given to various articles, typical examples of which are semiconductors such as a ULSI, without any thermal treatment. As a result, the performance or reliability thereof can greatly be improved. The aromatic ring polymer used in the invention appears to have a high strength since the polymer has the following molecular structure:

(1) Each molecular structure is rigid because of aromatic ring structures of the X and Y moieties in the formula (1) and the bonding stability of the A and B moieties therein.

(2) Aromatic ring π electron electrostatic interaction of the X and Y moieties in the formula (1) and intermolecular interaction, based on the twisted structure of the main chain are intense.

The strength of the material of the invention is varied in accordance with X, Y, A and B, which are main chain structures of the formula (1), the kind of the substituent(s) R, the substitution position thereof, the number of the substituent(s), the molecular weight, the molecular weight distribution, and so on. The hardness thereof according to the nano indentation method is preferably from 0.3 GPa to 30 GPa (inclusive), and/or the modulus thereof is from 3 GPa to 300 GPa (inclusive). More preferably, the hardness is from 0.4 GPa to 25 GPa (inclusive), and/or the modulus is from 4 GPa to 250 GPa (inclusive).

The definition of the modulus is as described in Evaluation Example 5.

For the aromatic ring polymer of the invention, the dielectric constant, the heat resistance or the strength thereof is improved by removing ionic impurities such as Fe³⁺, Cl⁻, Na⁺ and Ca²⁺, reaction solvent, post-treatment solvent, water content and so on by purification such as washing, ion exchange resin treatment, reprecipitation, recrystallization, microfiltration, or drying.

Usually, aromatic ring polymers are solvent-insoluble since the polymers are rigid. However, the aromatic ring polymer used in the present invention is soluble since the rigidity is appropriately lowered by the presence of A and B in the formula (1). The aromatic ring polymer can be made into a thin film since the polymer is amorphous. Accordingly, the polymer can be used as a heat resistant thin film for semiconductor devices, image display devices, electronic circuit devices, surface protecting films, and so on.

As the method for forming the thin film, a thin-film forming method such as spin coating, casting or bar coating can be preferably used. Conditions for forming the thin film are appropriately set since the solubility in solvent or the viscosity of the solution is varied with the kind of the substituent(s) R, the substitution position thereof, the number of the substituent (s), and so on. The solution is applied onto a desired surface by these methods, and subsequently the resultant is heated at a temperature over the boiling point or lower of the solvent under normal pressure or is heated at a temperature of the boiling point of the solvent under reduced pressure or under air flow of dry gas, thereby removing the solvent. In this way, a thin film can easily be formed. It is unnecessary to conduct high-temperature thermal treatment, which is necessary for thermally crosslinking material, after the removal of the solvent. However, in the case of making the strength higher or adjusting other properties, additives known in the prior art, such as a crosslinking agent, may be appropriately added.

The thin film made of the aromatic ring polymer of the invention is not required to be polymerized at high temperature (thermally cured) after the polymer has made into the film, and further the polymer has a simple chemical structure and can be produced from inexpensive raw materials. Accordingly, the film is economical and further the film can be favorably used since a catalyst and crosslinking agent necessary for thermally curing are unnecessary and the remaining thereof is never caused.

The film thickness, which is varied in accordance with intended applications, is preferably from 20 nm to 10 μm. The film thickness can be optically measured with an ellipsometer or the like, or can be mechanically measured with a stylus type film thickness measuring device, an AFM or the like.

A paint where the aromatic ring polymer of the invention is dissolved in an organic solvent can be used as a surface protecting film in the state that the paint is applied onto a painted surface or the surface of a plastic product. Examples of the solvent includes esters such as ethyl acetate and ethyl lactate, ethers such as anisole, amides such as NMP and DMF, aromatic solvents such as nitrobenzene and toluene, halogen solvents such as chloroform, dichloromethane and trichloroethane, and DMSO. For example, when this paint is applied onto a painted surface or the surface of a plastic product and subsequently the organic solvent is evaporated, a painted surface protecting film or a plastic hard coat film can be formed.

The aromatic ring polymer of the invention can be favorably used in various fields of fiber, molded bodies and others, as well as the above-mentioned articles because of its excellent properties. The aromatic ring polymer is used as, for example, a sheet, a tube, a film, fiber, a laminated product, a coating material, or various containers, or can be used for various parts, for example, mechanical parts, automobile parts (such as exterior parts such as a bumper, a fender, an apron, a hood panel, a fascia, a locker panel, a locker panel reinforcement, a floor panel, a rear quarter panel, a door panel, a door support, a roof top, a trunk lid and a fuel lid; interior parts such as an instrument panel, a console box, a glove box, a shift knob, a pillar garnish, a door trim, a handle, an arm rest, a window louver, a head rest, a seat belt, and a seat; engine room inner parts, such as a distributor cap, an air cleaner, a radiator tank, a battery case, a radiator shroud, a washer tank, a cooling fan, and a heater case; a mirror body, a wheel cover, a trunk mat, and a gasoline tank), two-wheeled vehicle parts (such as a cowling material, a muffler cover, and a leg shield), and electrical or electric parts (such as a housing, a chassis, a connector, a printed board, a pulley, air-conditioner parts, typewriter parts, word processor parts, camera parts, office computer related parts, telephone related parts, facsimile related parts, and copying machine related parts). The aromatic ring polymer is useful as material for various optical instruments, such as various lenses, prisms, optical fibers, optical discs, and liquid crystal panels.

### Embodiment 1

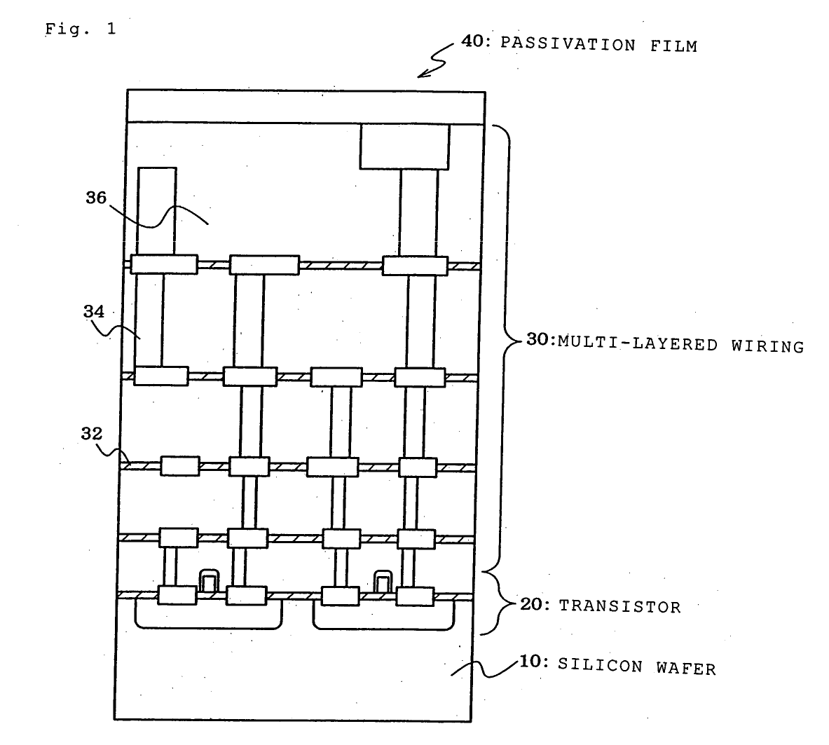

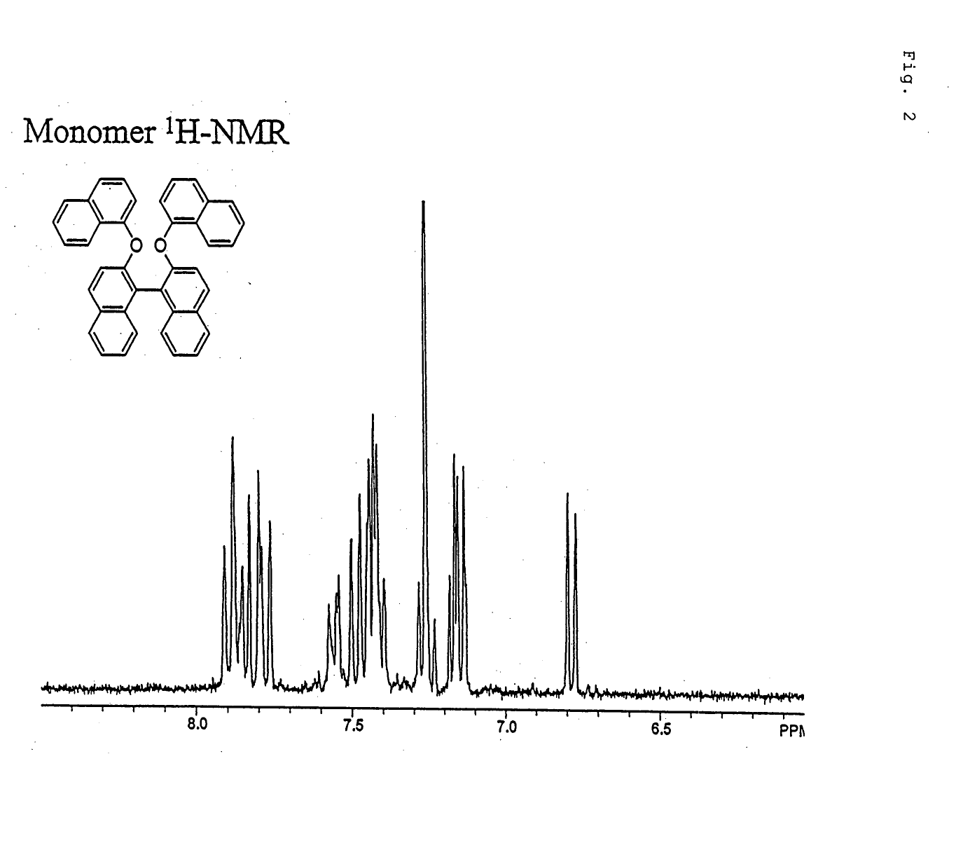

FIG. 1 illustrates an embodiment of a semiconductor device including an interlayer dielectric film made of the aromatic ring polymer of the invention.

An ultra large scale integration (ULSI) multi-layered wiring structure as illustrated in this figure, which is a kind of semiconductor device, comprises a silicon wafer 10, transistors 20, a multi-layered wiring 30, and a passivation film 40. A plurality of the multi-layered wirings 30 are multi-layered, thereby attaining high integration. The multi-layered wiring 30 is composed of Cu wires 34 for connecting hard masks and/or barrier metals 32, and interlayer dielectric film 36 present between the Cu wires 34. The interlayer dielectric film 36 is made of the aromatic ring polymer of the invention.

In this circuit, the dielectric constant of the aromatic ring polymer which constitutes the interlayer dielectric film 36 is low; therefore, even if the wiring working size (the interval between the Cu wires 34) is made narrow, electric charges are not easily parasitic between the Cu wires 34 so that the wiring delay time and/or power consumption can be controlled into a small value.

The heat resistance of the aromatic ring polymer which constitutes the interlayer dielectric film 36 is high. Accordingly, problems such as a breakdown thereof due to heat, a variation in the size, the generation of gas, and a deterioration, can be avoided when a semiconductor device is produced by way of fine processing techniques, for example, photolithography, etching, Cu wiring formation, vapor deposition, sputtering, and other processes exposed to high temperature.

Furthermore, the strength of the aromatic ring polymer which constitutes the interlayer dielectric film 36 is high. Accordingly, problems, such as a breakdown, damage, exfoliation or peeling thereof, can be avoided when a semiconductor device is produced by fine processing techniques, for example, photolithography, etching, Cu wiring formation, CMP (chemical mechanical polishing), vapor deposition, and sputtering.

### EXAMPLES

The present invention is not limited by the following examples. Catalysts, and chemical-agents used in Production Examples and Examples were commercially available products or products prepared in accordance with methods described in known literatures.

Any dipole moment and density in Examples were obtained by the above-mentioned methods.

### Production Example 1

[Synthesis of a Monomer for Aromatic Ring Polymer]

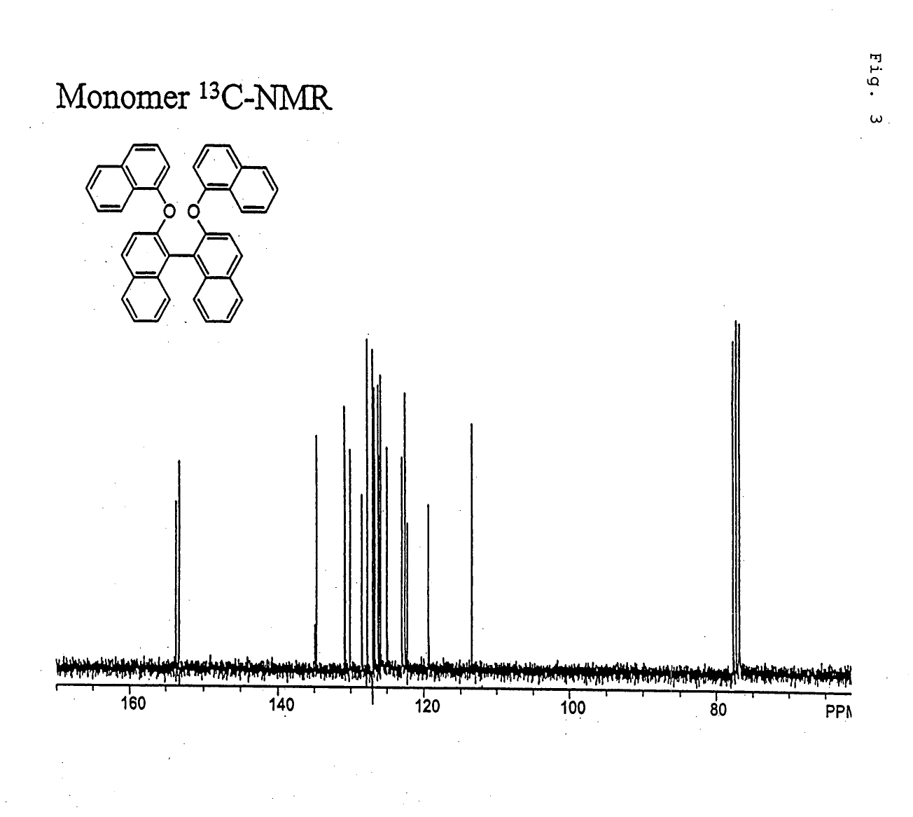

2,2′-Dihydroxy-1,1′-binaphthyl (1.15 g, 4 mmol) was charged into a flask of 50 mL volume in which toluene (10 mL) was put, and dissolved therein. Thereafter, quinoline (10 mL) was added thereto. A suspension in which potassium carbonate (1.38 g, 10 mmol) was added to this solution was heated at 150° C. in an oil bath while stirred. In this way, toluene was distilled off and further a very small amount of water contained in the system was removed by azeotropy. The system was naturally cooled up to room temperature, and then copper powder (0.026 g, 0.4 mmol) and 1-bromonaphthalene (1.12 mL, 8 mmol) were added thereto. The resultant was heated at 200° C. in an oil bath for 48 hours while stirred. The system was naturally cooled to room temperature. Thereafter, methylene chloride (20 mL) was added thereto, and the resultant was washed with diluted hydrochloric acid (1 N), and next washed with an aqueous NaOH solution (3%). Methylene chloride was distilled off under reduced pressure, thereby yielding a solid matter. THF (5 mL) was added thereto, thereby dissolving the solid matter into THF, and then an excessive amount of methanol was added thereto, thereby reprecipitating a solid matter. The resultant was filtrated so as to yield a crude product. This crude product was dissolved into methylene chloride (2 mL), and then purified by chromatography (toluene:hexane=1:2) using a silica gel column. Furthermore, the resultant was subjected to recrystallization using cyclohexane, thereby yielding 2,2′-dinaphthyloxy-1,1′-binaphthyl (1.12 g, yield: 52%). The structure thereof was checked through ¹H-NMR (FIG. 2) and ¹³C-NMR (FIG. 3).

### Production Example 2

[Synthesis of a Monomer for Dinaphthylamine Polymer]

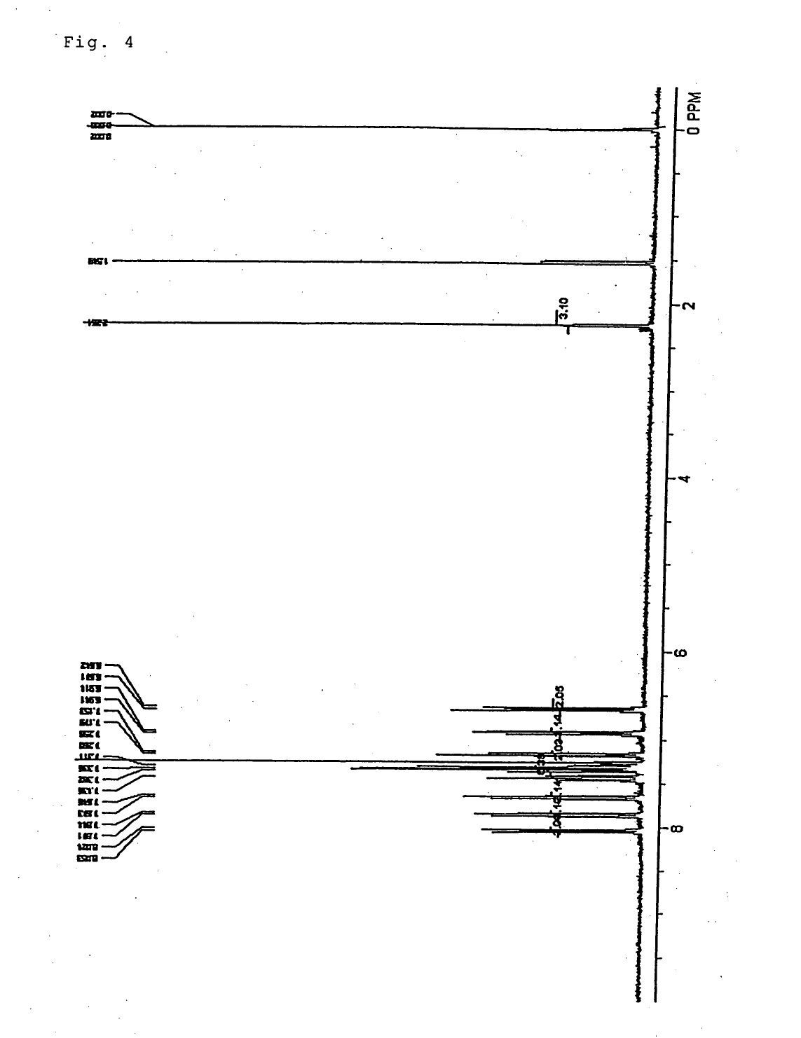

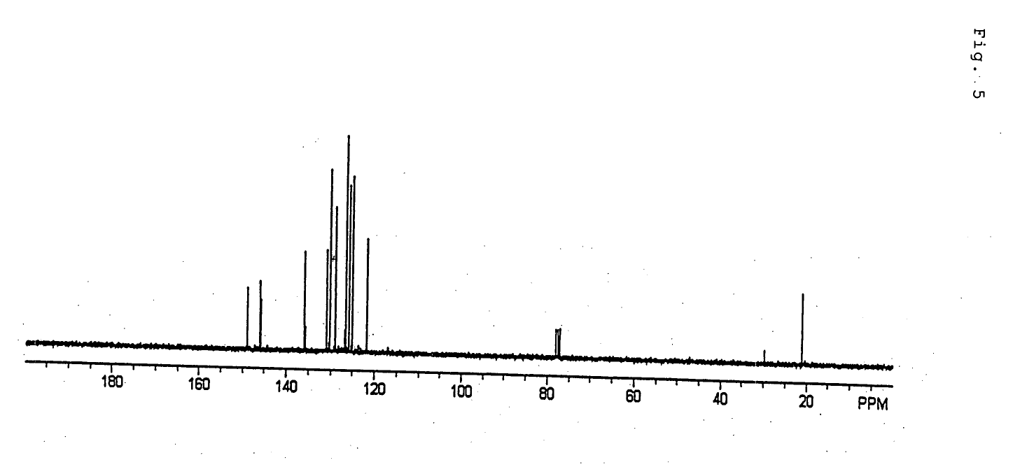

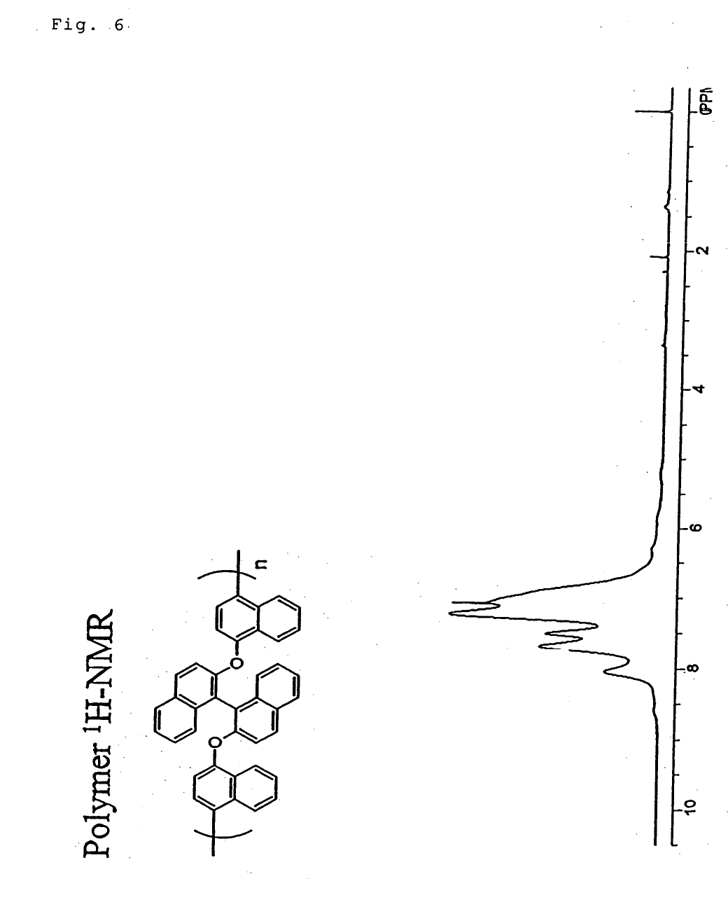

Into a 200 mL two-necked flask equipped with a cooling tube and a septum rubber were charged 1.6 g (15 mmol) of p-toluidine, 6.83 g (33 mmol) of 1-bromonaphthalene, 0.34 g (0.33 mmol) of Pd₂(dba)₃, 4.44 g (46.2 mmol) of t-BuONa, and a stirring bar. The inside thereof was purged with nitrogen. When the purging was sufficiently completed, a syringe was used to add 100 mL of distilled toluene thereto. Next, a syringe was used to add 0.357 mL (1.32 mmol) of P(t-Bu)₃ thereto. The solution was heated at 80° C. for 6 hours while stirred. It was checked whether or not the raw materials were consumed by TLC, and a precipitation was separated by filtration. The filtrate was concentrated, and purified by silica gel column chromatography (CHCl₃) Subsequently, the resultant was recrystallized from methanol/2-propanol, so as to yield di-(1-naphthyl)-4-toluylamine (DNTA) as a white solid. The yield was 88% (4.76 g). The structure thereof was checked through ¹H-NMR (FIG. 4) and ¹³C-NMR (FIG. 5).

### Example 1

[Synthesis of an Aromatic Ring Polymer]

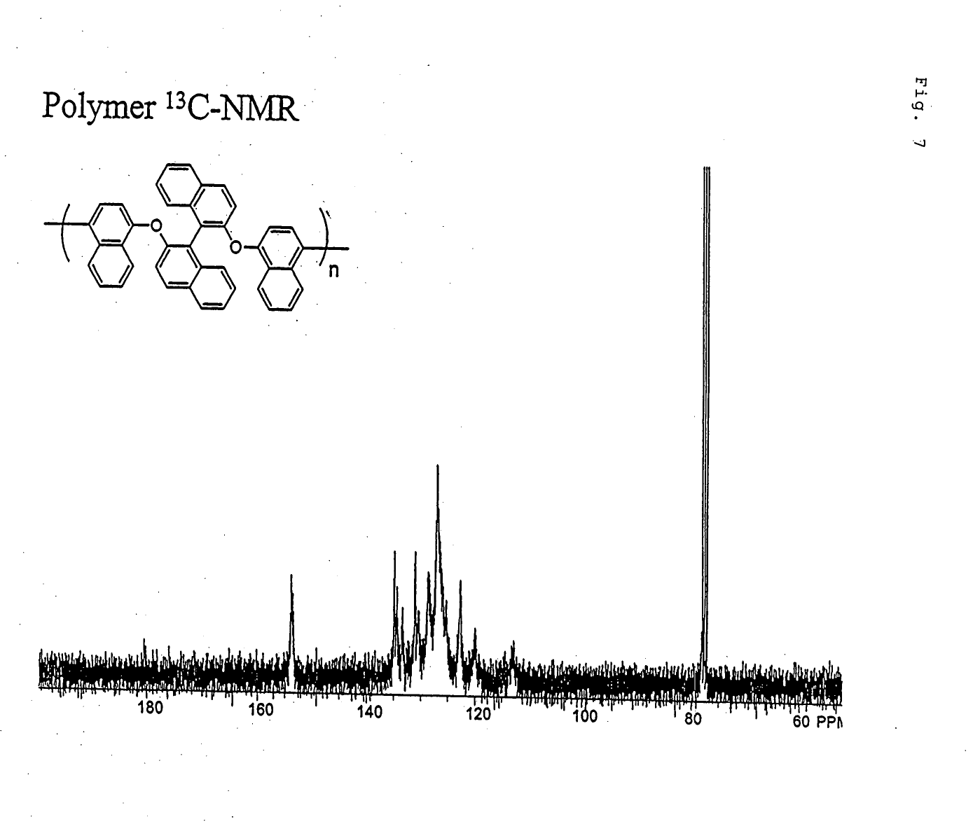

2,2′-Dinaphthyloxy-1,1′-binaphthyl (0.48 g, 0.9 mmol) synthesized in Production Example 1 was completely dissolved into a flask of 20 mL volume in which nitrobenzene (2.8 mL) was put, and then ferric chloride (0.49 g, 3 mmol) was added thereto. This suspension was stirred at room temperature, and caused to react for 24 hours. This polymer solution was poured into acidic methanol, thereby dissolving iron compounds containing ferric chloride and precipitating a polymer. This polymer was filtrated, dried under a reduced pressure, and then dissolved into chloroform (5 mL) to prepare a homogeneous solution. This homogeneous solution was poured into acetone (20 mL), and the resultant was filtrated, and dried under a reduced pressure to yield poly(2,2′-dinaphthyloxy 1,1′-binaphthyl) (0.45 g, yield: 94%). The structure thereof was checked through ¹H-NMR (FIG. 6) and ¹³C-NMR (FIG. 7). The molecular weight was measured by GPC (in terms of polystyrene, using chloroform as its mobile phase) (Mn=9500, Mw=28000).

It was verified from calculation based on the AM1 method that the adjacent naphthalene groups in the most stable structure were not on a single plane.

The dipole moment was 0.1 debye, and the density was 1.13 g/cm³.

### Example 2

[Purification of an Aromatic Ring Polymer]

A part (0.34 g) of poly(2,2′-dinaphthyloxy 1,1′-binaphthyl) synthesized in the same way as in Example 1 was dissolved into chloroform (100 mL). An ion exchange resin (Amberlight (registered trademark) EG-4-HG, manufactured by ORGANO Corporation) subjected beforehand to substitution treatment with chloroform was poured, in a volume of 50 mL, into the solution. At room temperature, the solution was stirred for 8 hours. The ion exchange resin was removed by filtration, and then the solution was concentrated under a reduced pressure, and poured into methanol. The precipitated solid was collected by filtration, and dried under a reduced pressure to yield poly(2,2′-dinaphthyloxy 1,1′-binaphthyl) treated with the ion exchange resin (0.31 g, yield: 91%).

### Example 3

[Synthesis of a Dinaphthylamine Polymer]

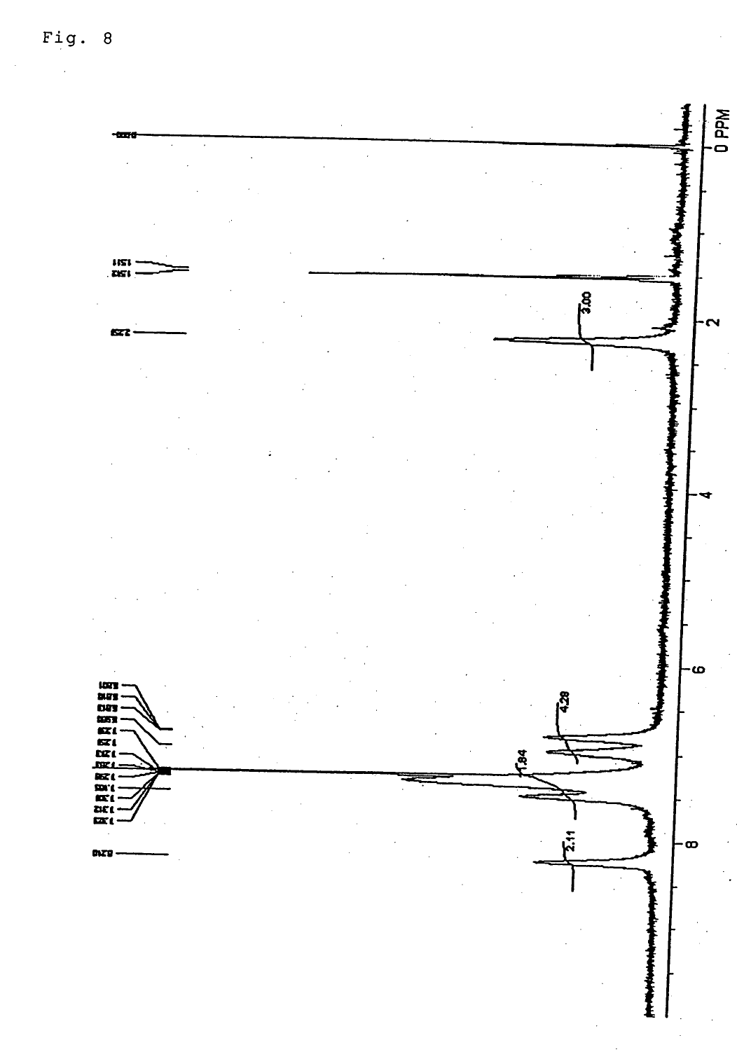

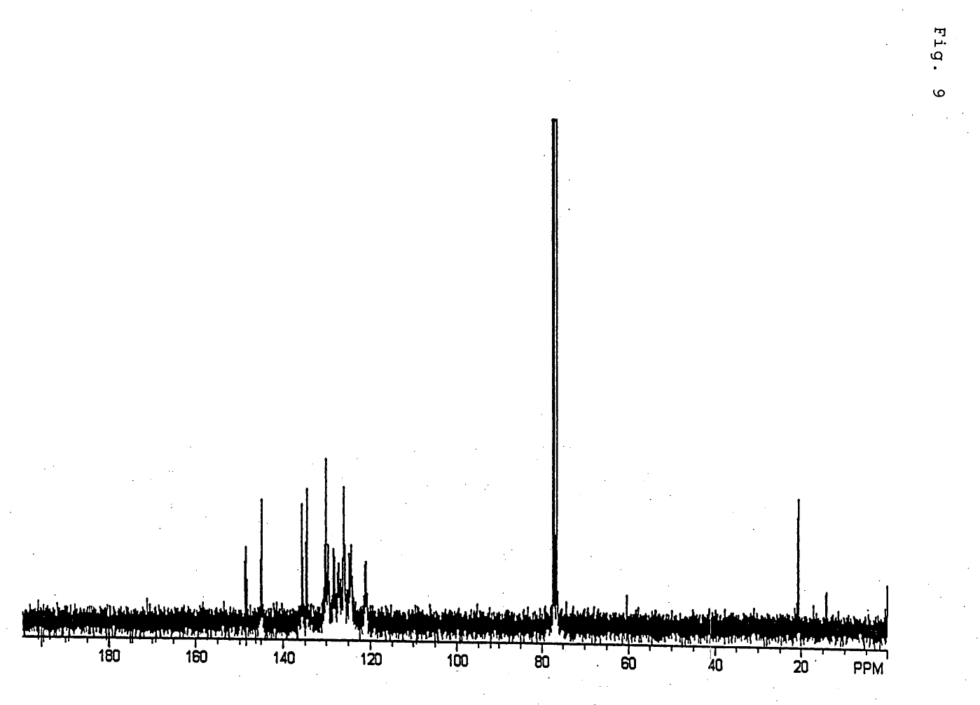

Into a 30-mL eggplant type flask were put a weighed amount of 0.178 g (0.5 mmol) of DNTA yielded in Production Example 2 and 1 mL of nitrobenzene, and the flask was degassed. After the flask was sufficiently degassed, 0.202 g (1.25 mmol) of FeCl₃ was quickly charged into the flask. The solution was stirred at room temperature for 24 hours. After the 24 hours, the resultant was poured into 100 mL of acidic methanol (1/10=HCl/MeOH) to precipitate a solid. The precipitated solid was separated by filtration, dissolved into THF, and poured into ammonia water and stirred. After the stirring for 30 minutes, a solid was separated by filtration, and dissolved into chloroform. This was poured into 100 mL of methanol, and the solution was stirred. The precipitated solid was separated by filtration, and dried under a reduced pressure to yield poly(di-(1-naphthyl)-4-toluylamine) as yellow ocher powder. The yield was 83% (0.146 g). The structure thereof was checked through ¹H-NMR (FIG. 8) and ¹³C-NMR (FIG. 9). The molecular weight was measured by GPC (in terms of polystyrene, using chloroform as its mobile phase) (Mn=13200, Mw/Mn=5.3).

It was verified from calculation based on the AM1 method that adjacent naphthalene groups in the most stable structure were not on a single plane.

The dipole moment was 0.4 debye, and the density was 1.10 g/cm³.

### Example 4

[Purification of a Dinaphthyl Amine Polymer]

A part (0.10 g) of poly(di-(1-naphthyl)-4-toluylamine) synthesized in the same way as in Example 3 was dissolved into tetrahydrofuran (100 mL). This solution was treated by passing the solution through a column tube filled with 100 mL of an ion exchange resin (Amberlight (registered trademark) 15J-HG-DRY, manufactured by ORGANO Corporation) subjected beforehand to substitution treatment with tetrahydrofuran. The resultant was concentrated under a reduced pressure, and poured into methanol. The precipitated solid was collected by filtration, and dried under a reduced pressure to yield poly(di-(1-naphthyl)-4-toluylamine) treated with the ion exchange resin (0.09 g, yield: 90%).

### Evaluation Example 1

[Evaluation as an Interlayer Dielectric Film]

Poly(2,2′-dinaphthyloxy-1,1′-binaphthyl) synthesized in Example 1 was used to prepare a solution thereof in nitrobenzene having a concentration of 15% by weight. This was applied onto a silicon wafer by use of a spin coater rotating at 3000 rpm for 20 seconds, so as to form a viscous thin film on the silicon wafer. The silicon wafer on which this viscous thin film was formed was heated at 150° C. for 5 minutes, thereby forming a non-viscous thin film having an even surface form. The thickness of this film was 0.44 μm according to a measurement with a stylus type film thickness measuring device.

About plural sites thereof, the dielectric constants were measured by the mercury probe method. The dielectric constants k were from 2.4 to 2.6. The 5% weight reduction temperature was 500° C. by thermogravimetry. The hardness was 0.4 GPa and the modulus was from 6.8 to 6.6 GPa according to the nano indentation method.

During the above-mentioned operation, it was never observed that any film was peeled from the silicon wafer. Thus, about the adhesive property to the substrate also, no problem was caused. The above-mentioned results demonstrated that the polymer can be favorably used as an interlayer dielectric film material for semiconductor.

### Evaluation Example 2

[Evaluation as an Interlayer Dielectric Film]

Poly(di-(1-naphthyl)-4-toluylamine) synthesized in Example 3 was used to form a non-viscous thin film in the same way as in Evaluation Example 1. The thickness of this film was 0.54 μm according to a measurement with the stylus type film thickness measuring device.

About plural sites thereof, the dielectric constants were measured by the mercury probe method. The dielectric constants k were from 2.5 to 2.7. The 1% weight reduction temperature was 448° C. by thermogravimetry. The hardness was 0.5 GPa and the modulus was 6.6 GPa according to the nano indentation method.

During the above-mentioned operation, it was never observed that any film was peeled from the silicon wafer. Thus, about the adhesive property to the substrate also, no problem was caused. The above-mentioned results demonstrated that the polymer can be favorably used as an interlayer dielectric film material for semiconductor.

### Evaluation Example 3

[Heat Resistance Evaluation]

Powder of poly(2,2′-dinaphthyloxy 1,1′-binaphthyl) synthesized in the same way in Example 1 was used. By means of a differential scanning calorimeter (DSC) and a thermogravimetry differential thermal analysis (Tg/DTA) (EXSTAR 6000, manufactured by Seiko Instruments Inc.), the DSC and the Tg/DTA were analyzed under temperature-raising conditions of 5° C./minute and 10° C./minute, respectively. As a result, the glass transition temperature (Tg), the 1% weight reduction temperature (T_{d1}), and the 5% weight reduction temperature (T_{d5}) were measured. Tg was 301° C., T_{d1} was 418° C., and T_{d5} was 520° C. The above-mentioned results demonstrated that the polymer can be favorably used as a high heat-resistant material.

### Evaluation Example 4

[Heat Resistance Evaluation]

An evaluation was carried out in the same way as in Evaluation Example 3 except that poly(di-(1-naphthyl)-4-toluylamine) synthesized in Example 3 was used instead of poly(2,2′-dinaphthyloxy-1,1′-binaphthyl) in Evaluation Example 3. As a result, no Tg was observed, and T_{d1} and T_{d5} were 448° C. and 538° C., respectively. The above-mentioned results demonstrated that the polymer can be favorably used as a high heat-resistant material.

### Comparative Example 1

An evaluation was carried out in the same way as in Evaluation Example 3 except that commercially available poly(2,6-dimethyl-1,4-phenyleneoxide) (manufactured by SIGMA-ALDRICH Corp.) was used instead of poly(2,2′-dinaphthyloxy-1,1′-binaphthyl) in Evaluation Example 3. As a result, Tg was 211° C., and the polymer started to be melted at 268° C. The polymer was a viscous liquid at the temperature or higher temperatures. The above-mentioned results demonstrated that the polymer is unsatisfactory as a highly heat-resistant material.

### Evaluation Example 5

[Strength Evaluation]

Poly(2,2′-dinaphthyloxy-1,1′-binaphthyl) synthesized in Example 2 was used to prepare a solution thereof in nitrobenzene having a concentration of 15% by weight. This was applied onto a silicon wafer by use of a spin coater rotating at 3000 rpm for 20 seconds, so as to form a viscous thin film on the silicon wafer. The silicon wafer on which this viscous thin film was formed was heated at 150° C. for 5 minutes, thereby forming a non-viscous thin film having an even surface form. The thickness of this film was 0.44 μm according to a measurement with the stylus type film thickness measuring device. This thin film was measured by the nano indentation method. As a result, the hardness was 0.42 GPa and the modulus was 9.8 GPa. The device for the measurement was a Triboscope system (trade name) (manufactured by Hysitron Inc.), and the used indenter (diamond) was in the form of a triangular pyramid. The modulus Er (complex modulus) was obtained from the following equation:
1/Er={(1−vs²)/Es+{(1−vi²)/Ei}
wherein Es is the Young's modulus of a sample, vs is the Poisson's ratio of the sample, Ei is the Young's modulus of the indenter, and vi is the Poisson's ratio of the indenter.

During the above-mentioned operation, it was never observed that any film was peeled from the silicon wafer. Thus, about the adhesive property to the substrate also, no problem was caused. The above-mentioned results demonstrated that the polymer can be favorably used as a high strength thin film material.

About poly(2,2′-dinaphthyloxy-1,1′-binaphthyl) yielded in Example 1 also, the same measurements were made. As a result, the film thickness was 0.47 μm, the hardness was 0.4 GPa, and the modulus was 6.8 GPa.

### Evaluation Example 6

[Strength Evaluation].

An evaluation was carried out in the same way as in Evaluation Example 5 except that poly(di-(1-naphthyl)-4-toluylamine) yielded in Example 4 was used instead of poly(2,2′-dinaphthyloxy-1,1′-binaphthyl) in Evaluation Example 5. The film thickness was 0.18 μm according to measurement with the stylus type film thickness measuring device. This thin film was measured by the nano indentation method. As a result, the hardness was 0.49 GPa and the modulus was 8.0 GPa.

During the above-mentioned operation, it was never observed that any film was peeled from the silicon wafer. Thus, about the adhesive property to the substrate also, no problem was caused. The above-mentioned results demonstrated that the polymer can be favorably used as a high strength thin film material.

About poly(di-(1-naphthyl)4-toluylamine) yielded in Example 3 also, the same measurements were made. As a result, the film thickness was 0.54 μm, the hardness was 0.5 GPa, and the modulus was 6.6 GPa.

### Comparative Example 2

An evaluation was carried out in the same way as in Evaluation Example 5 except that commercially available poly(2,6-dimethyl-1,4-phenyleneoxide) (manufactured by SIGMA-ALDRICH Corp.) was used instead of poly(2,2′-dinaphthyloxy-1,1′-binaphthyl) in Evaluation Example 5. The film thickness was 0.38 μm according to measurement with the stylus type film thickness measuring device. This thin film was measured by the nano indentation method. As a result, the hardness was 0.16 GPa and the modulus was 3.5 GPa. During the above-mentioned operation, it was observed that a film was peeled from the silicon wafer. Thus, it was demonstrated that the polymer is unsatisfactory as a high strength thin film material, for example, the adhesive property to the substrate is unsatisfactory.

### INDUSTRIAL APPLICABILITY

The present invention can provide a new aromatic ring polymer and an excellent low dielectric material.

The low dielectric material made of the aromatic ring polymer of the invention can be used as an interlayer dielectric film material without introducing any pore into the material, and makes it possible to improve the performance of semiconductors, such as a ULSI, greatly.

The invention can provide a heat resistant material which can exhibit a high heat resistance without undergoing any thermal crosslinking.

The invention can provide a high strength material which can exhibit a high strength without undergoing any thermal crosslinking.

1. An aromatic ring polymer, comprising a main chain in which aromatic rings range, the aromatic ring polymer having a dipole moment of 1 debye or less and/or a density of 1.50 g/cm³ or less by cancellation of the dipole moments of the aromatic rings in the most stable structure thereof.

2. An aromatic ring polymer, wherein adjacent aromatic ring skeletons cannot have a conformation positioned on a single plane due to mutual steric hindrance of the adjacent aromatic ring skeletons and is represented by the following formula (1): [symbol]
X-A-Y-B [symbol] ₙ (1)
wherein X and Y may be the same or different, and each represent a monocyclic or heterocyclic aromatic group which may be substituted with R;
A and B may be the same or different, and each represent a single bond, a bifunctional substituent selected from —(CR₂)ₘ, —(SiR₂)ₘ—, —(OSiR₂O)ₘ—, —(SiRO_{1.5})ₘ—, —(GeR₂)ₘ—, —(SnR₂)ₘ—, —BR—, —AlR—, —NR—, —PR—, —AsR—, —SbR—, —O—, —S—, —Se—, —Te—, —CO—, —COO—, —OO—, —NHCO—, —(N═C)—, an acetylidene group, an ethylidene group, a borazilene group, a substituted or unsubstituted aromatic group having 6 to 50 carbon atoms and a substituted or unsubstituted heteroatom-containing aromatic group having 4 to 50 carbon atoms, or a substituent formed by combining one or more out of these substituents;
R may be the same or different, and each represent an alkyl group having 1 to 20 carbon atoms, an alkenyl group having 1 to 20 carbon atoms, an alkynyl group having 1 to 20 carbon atoms, a substituted or unsubstituted aromatic group having 6 to 20 carbon atoms, an ether group, a thioether group, an ester group, an epoxy-containing group, a silyl-containing group, a siloxy-containing group, a fluorine-containing group, a borazyl group, or a substituent formed by combining two or more out of these substituents;
m is an integer of 1 to 50; and
n is an integer of 5 to 1000000.

3. An aromatic ring polymer, wherein adjacent aromatic ring skeletons cannot have a conformation positioned on a single plane due to mutual steric hindrance of the adjacent aromatic ring skeletons and is represented by the following formula (2): [symbol]
X-A′-Y [symbol] (2)
wherein X and Y may be the same or different, and each represent a monocyclic or heterocyclic aromatic group which may be substituted with R;
A′ may be the same or different, and each represent a monocyclic or heterocyclic aromatic group which may be substituted with R and is bonded to X and Y through any one of oxygen, nitrogen, sulfur, silicon and boron or through a substituent containing one or more out of these atoms;
R may be the same or different, and each represent an alkyl group having 1 to 20 carbon atoms, an alkenyl group having 1 to 20 carbon atoms, an alkynyl group having 1 to 20 carbon atoms, a substituted or unsubstituted aromatic group having 6 to 20 carbon atoms, an ether group, a thioether group, an ester group, an epoxy-containing group, a silyl-containing group, a siloxy-containing group, a fluorine-containing group, or a substituent formed by combining two or more out of these substituents; and
n is an integer of 5 to 1000000.

4. An aromatic ring polymer represented by the following formula (3): 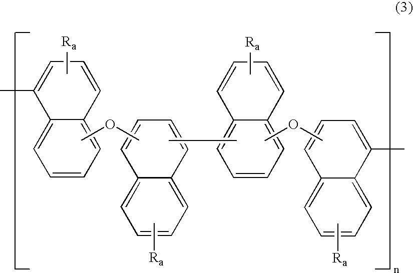
wherein each R and n are the same as in the formula (1), and a's may be the same or different, and are each an integer of 0 to 6.

5. An aromatic ring polymer represented by the following formula (4): 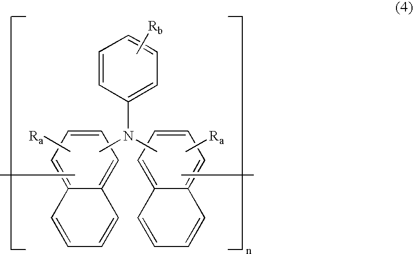
wherein each R and n are the same as in the formula (1), a's may be the same or different, and are each an integer of 0 to 6, and b's may be the same or different, and are each integer of 0 to 5.

6. The aromatic ring polymer according to claim 2, which has a dipole moment of 1 debye or less, and/or a density of 1.20 g/cm³ or less.

7. A low dielectric material, comprising the aromatic ring polymer according to claim 1.

8. An interlayer dielectric film material for semiconductor, comprising the low dielectric material according to claim 7.

9. A heat resistant material, comprising the aromatic ring polymer according to claim 1.

10. The heat resistant material according to claim 9, the glass transition temperature of the heat resistant material being 250° C. or higher, and a lower temperature out of the melting temperature thereof or the thermal decomposition starting temperature thereof being 300° C. or higher.

11. A high strength material, comprising the aromatic ring polymer according to claim 1.

12. The high strength material according to claim 11, which has a hardness of 0.3 GPa or more, and/or a modulus of 3 GPa or more.

13. A thin film, comprising the aromatic ring polymer according to claim 1.

14. A semiconductor device, comprising the thin film according to claim 13.

15. An image display device, comprising the thin film according to claim 13.

16. An electronic circuit device, comprising the thin film according to claim 13.

17. A surface protecting film, comprising the thin film according to claim 13.

18. A paint wherein the aromatic ring polymer according to claim 1 is dissolved in an organic solvent.
